(19) United States
(12) Patent Application Publication
(10) Pub. No.: US 2024/0161936 A1
(43) Pub. Date: May 16, 2024

(54) Medical Video Charting Application

(71) Applicant: ShasKEYNOTE, LLC., Salt Lake City, UT (US)

(72) Inventor: David Shaskey, Salt Lake City, UT (US)

(21) Appl. No.: 18/054,669

(22) Filed: Nov. 11, 2022

(51) Int. Cl. G16H 80/00

(52) U.S. Cl. CPC G16H 80/00

(57) ABSTRACT

Methods and equipment for medical video charting. Some embodiments of the present invention may comprise a kit comprising a web-enabled camera, laptop, access to a cloud-based storage system, and a set of instructions for a method for medical video charting.

### CROSS-REFERENCES TO RELATED APPLICATIONS

Not Applicable.

### FIELD OF INVENTION

The present invention relates generally to a computer application for documentation and physician/medical encounters. With this app the provider does NOT have to primarily access the computer or EMR (Electronic Medical Record), thereby allowing a lower level assistant to do/finish the charting, allowing the provider to have a much greater amount of time for direct patient care and also as a result to more accurately document the details—even dramatically reducing the malpractice and untoward event risk BACKGROUND

Currently physicians document/chart patient encounters on one of a variety of computer programs, all of which are inherently time intensive for the physician. A typical doctor spends 2-3 hours each day on the computer documenting visits. This is a heinous task that takes time away from direct patient care, that is the true life cblood of all physicians. Many physicians hate this task even more due to limited computer/typing skills making it even more difficult to finish the necessary but imperative task.

It may, therefore, be advantageous to provide an improved method and application for charting and recording medical information following appointments and other encounters.

### SUMMARY

This Summary is provided to introduce a selection of concepts in a simplified form that is further described below in the Detailed Description. This Summary is not intended to identify key features or essential features of the claimed subject matter, nor is it intended to be used as an aid in determining the scope of the claimed subject matter. The present invention is defined by the claims.

Aspects of the present invention are directed towards methods and equipment for medical video charting. Some embodiments of the present invention may comprise a kit comprising a lap top, a web-enabled camera, access to a cloud based storage system, and a set of instructions for a method for medical video charting.

### BRIEF DESCRIPTION OF THE DRAWINGS

The present invention is described in detail below with reference to the attached drawing FIGURES, wherein:

FIG. 1 provides a flowchart of an exemplary method is accordance with aspects of the present invention.

### DETAILED DESCRIPTION

Some embodiments of the present invention are described with specificity herein to meet statutory requirements. However, the scope of the invention is not intended to be defined by the description itself. The claimed subject matter may be embodied as to include different features, elements, components, steps, or combinations of steps, similar to those described herein, and in combination with other existing or future technologies. Moreover, although the term “step” might be used to connote different elements of the methods employed, this term should not be interpreted as implying any particular order among or between various steps herein disclosed unless and except the order of individual steps is explicitly described or required.

Aspects of the present invention are directed towards methods and equipment for medical video charting. Some embodiments of the present invention may comprise a kit comprising a lap top, a web-enabled camera, access to a cloud based storage system, and a set of instructions for a method for medical video charting.

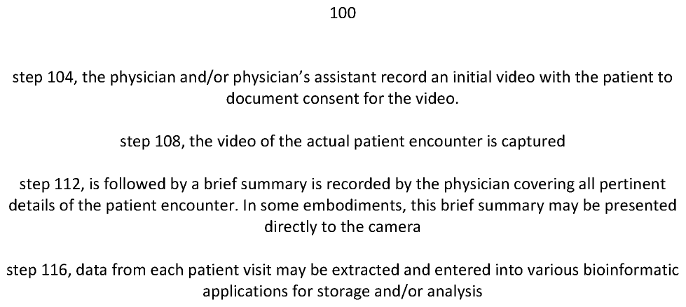

FIG. 1 provides a flowchart 100 of an exemplary method is accordance with aspects of the present invention. At step 104, the physician and/or physician's assistant record an initial video with the patient to document consent for the video. At step 108, the video of the actual patient encounter is captured. The term “patient encounter”, as used herein is a broad term which may include but is not limited to, routine check-ups, urgent care appointments, in-patient rounds, telehealth consultations, and the like. The patient encounter may comprise but not be limited to review of patient medical history, physical examination, discussion and/or consultation with patient and/or the patient's family members regarding subjects including but not limited to diagnosis, evaluation, treatment, and follow-up recommendations and the like. At step 112, is followed by a brief summary is recorded by the physician covering all pertinent details of the patient encounter. In some embodiments, this brief summary may be presented directly to the camera. This represents a complete patient encounter and requires no additional record keeping or charting on the part of the physician to document the visit.

After the patient encounter, at step 116, data from each patient visit may be extracted and entered into various bioinformatic applications for storage and/or analysis. In some embodiments, data may be extracted from the video record of the patient encounter via an artificial intelligence enabled transcription application. In other embodiments, data may be extracted manually by the physician's assistant and/or by the physician. In some embodiments, transcriptions may be delayed for a later time for more efficient time use. At step 120, the video of the complete patient encounter may be archived for later review if necessary.

Implementation of such a method as described herein may save each physician implementing it an average of 2 to 3 hours per day and may represent a significant improvement to the workflow efficiency of such a physician. A lower level assistant, with minimal training can complete the days encounters in a very brief time-minutes to not much more than an hour-a dramatic improvement in the efficiency of the provider's time.

Embodiments of the present invention have been described, as required by statute, to be illustrative, but should not be interpreted to be restrictive. One having skill in the art will recognize that many different arrangements of the various components depicted are possible without departing from the scope of the claims below, as well as arrangements including components not explicitly shown.

One having skill in the art will understand that certain combinations and/or sub-combinations of elements and features are of utility and may be employed without reference to other combinations and/or sub-combinations and are contemplated within the scope of the claims. Not all steps listed in the various FIGURES need be carried out in the specific order described.

What is claimed is:

1. A method for medical video charting comprising:
recording an initial video with a patient to document the patient's consent to video medical charting;
capturing a video of a patient encounter;
recording a brief summary of the patient encounter covering all pertinent details of the patient encounter;
extracting data from the patient encounter into at least one bioinformatic application for storage and analysis; and
archiving the video of the patient encounter for later review.

2. The method of independent claim 1, wherein:
the patient encounter comprises at least of on a routine check-up, urgent care appointment, in-patient round, and telehealth consultation.

3. The method of independent claim 1, wherein:
the patient encounter comprises at least one of a review of patient medical history, physical examination, and consultation.

4. The method of independent claim 1, wherein:
the brief summary is presented directly to the camera.

5. The method of independent claim 1, wherein:
data may be extracted from the video record of the patient encounter to an easily trainable assistant or via an artificial intelligence enabled transcription application.

6. The method of independent claim 1, wherein:
data is extracted manually by at least one of a physician's assistant and a physician.

7. A composition of matter for medical video charting comprising:
a laptop; a web-enabled camera;
cloud-based media storage; and
a set of instructions for a method of medical video charting, the method comprising:
recording an initial video with a patient to document eth patient's consent to video medical charting;
capturing a video of a patient encounter;
recording a brief summary of the patient encounter covering all pertinent details of the patient encounter;
extracting data from each patient visit into at least one bioinformatic application for storage and analysis; and
archiving the video of the patient encounter for later review.
